(19) United States
(12) Patent Application Publication
(10) Pub. No.: US 2022/0251112 A1
(43) Pub. Date: Aug. 11, 2022

(54) PROCESS FOR PREPARATION OF EDOXABAN

(71) Applicant: Glenmark Life Sciences Limited, Mumbai (IN)

(72) Inventors: Prem Chand, Mumbai (IN); Pramod Vitthal Patil, Panvel (IN); Balasahab Jadhav, Pune (IN); Sachin Mahadeo Lad, Thane (IN); Samir Naik, Thane (IN); Shekhar Bhaskar Bhirud, Mumbai (IN); Nasir Ali Shafakat Ali, Thane (IN)

(21) Appl. No.: 17/624,595

(22) PCT Filed: Jun. 24, 2020
(86) PCT No.: PCT/IB2020/055950

§ 371 Date: Jan. 4, 2022

(30) Foreign Application Priority Data
Jul. 4, 2019 (IN) 201921026759

(51) Int. Cl. C07D 513/04

(52) U.S. Cl. CPC C07D 513/04

(57) ABSTRACT

The present invention relates to process for preparation of N1-(5-Chloropyridin-2-yl)-N2-[(1S,2R,4S)-4-(N,N-dimethylcarbamoyl)-2-(5-methyl-4,5,6,7-tetrahydrothiazolo [5,4-c]pyridin-2-ylcarboxamido)cyclohexyl]oxamide p-toluene sulfonate monohydrate [edoxaban tosylate monohydrate], the compound of formula I, comprising reacting compound of formula VI with compound of formula V to obtain the compound of formula IV and further converting it to edoxaban tosylate monohydrate in an industrially feasible process.

### PRIORITY

This application claims the benefit of Indian Provisional Application 201921026759 filed on Jul. 4, 2019, entitled “PROCESS FOR PREPARATION OF EDOXABAN”, the contents of which are incorporated herein by reference.

### FIELD OF THE INVENTION

The present invention relates to a process for the preparation of edoxaban and salts thereof.

### BACKGROUND OF THE INVENTION

Savaysa (Edoxaban tosylate monohydrate) is N-(5-chloropyridin-2-yl)-N-[(1S,2R,4S)-4 (N,N-dimethyl carbamoyl)-2-(5-methyl-4,5,6,7-tetrahydro [1,3]thiazolo[5,4-c]pyridine-2-carboxamido)cyclohexyl]oxamide mono(4-methyl benzenesulfonate) monohydrate depicted by compound of formula I.

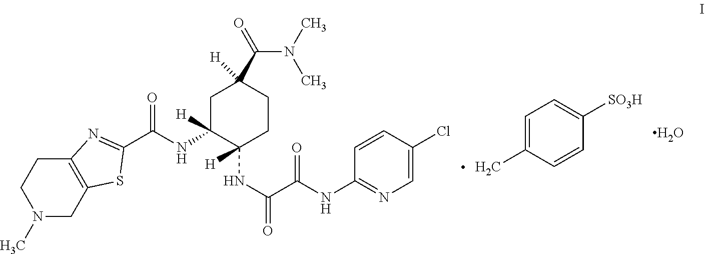

Savaysa is a factor Xa inhibitor, indicated to reduce the risk of stroke and systemic embolism (SE) in patients with nonvalvular atrial fibrillation (NVAF). Savaysa is available as 15 mg, 30 mg and 60 mg oral tablets.

The present invention provides a simple and industrially advantageous process for preparing edoxaban. In the preparation of edoxaban, compound of Formula IV is an important intermediate, prepared by the reaction of compound of formula V with a compound of formula VI.

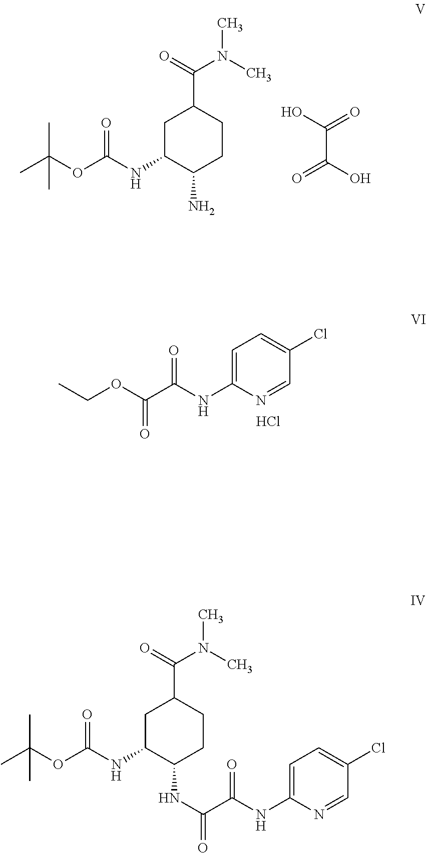

This reaction requires use of a base and it has been observed that when a base is added to the compound of formula V, and when acetonitrile is used as a solvent there is formation of a gel which gets deposited and consequently the reaction system hardens and solidifies making stirring difficult and reducing the yield and purity of compound of formula IV. To circumvent this problem, the compound of formula VI is treated with one portion of a base and then the compound of formula V is added followed by addition of another portion of the base. Thus a particular sequence of addition of the two reactants compound V and VI and adding the base in divided portions is required to solve the problem of the reaction system solidifying.

Surprisingly it has been found that by the process of the present invention, wherein dimethyl sulfoxide is used as a solvent in the reaction of compound of formula V with a compound of formula VI in presence of a base, the problem of gel formation and reaction system hardening is circumvented, leading to the formation of compound of formula II in high yields and purity. The use of dimethylsulfoxide as a solvent does not require a particular sequence of addition of reactants or use of base in divided portions and provides reaction simplicity and also ensures the completion of reaction in lesser time.

### SUMMARY OF THE INVENTION

The present invention provides a process for the preparation of edoxaban tosylate monohydrate, a compound of formula I, comprising:

a) reacting the compound of formula V with a compound of formula VI in the presence of a base and a solvent to obtain compound of formula IV, wherein the solvent is dimethyl sulfoxide;

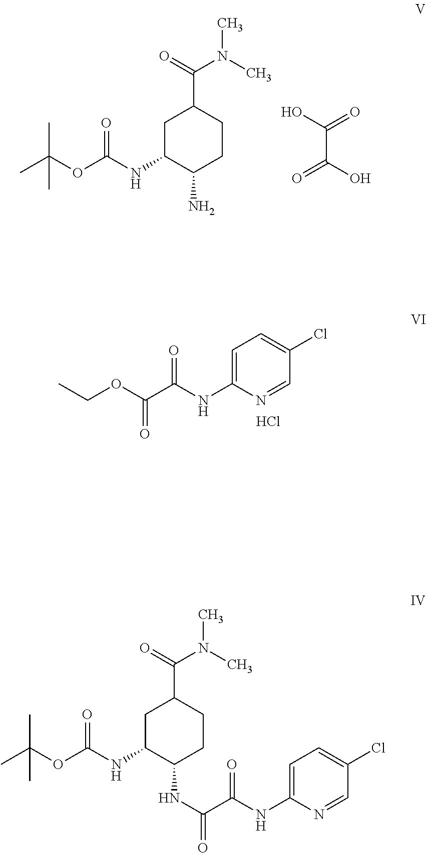

b) reacting the compound of formula IV with a deprotecting reagent to obtain a compound of formula II or salt thereof;

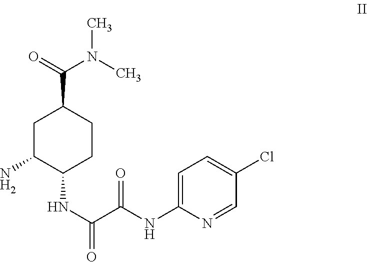 [SMILES: CN(C)C(=O)[C@H]1CC[C@H](NC(=O)C(=O)Nc2ccc(Cl)cn2)[C@H](N)C1]

c) reacting the compound of formula II or salt thereof with compound of formula III in the presence of a base to obtain a compound of formula I-A;

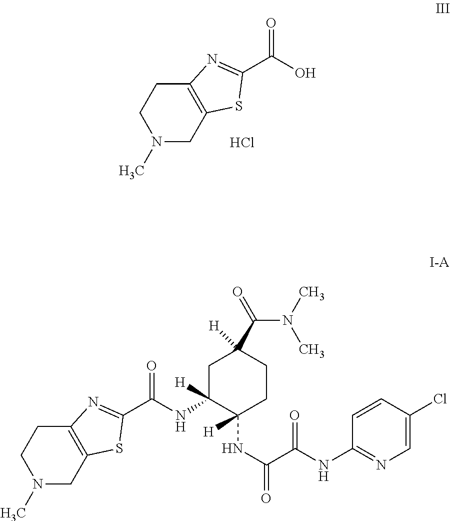

d) reacting the compound of formula I-A with para toluene sulfonic acid in a solvent to obtain a compound of formula I; and
e) optionally, recrystallizing the compound of formula I with a solvent.

### BRIEF DESCRIPTION OF THE DRAWINGS

FIG. 1: XRD pattern of edoxaban tosylate monohydrate as obtained from example 4E.

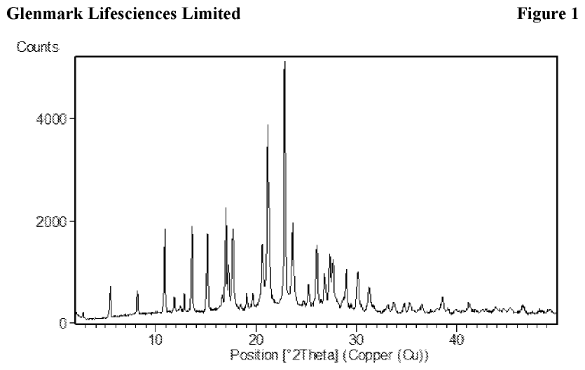

FIG. 2: XRD pattern of edoxaban free base as obtained from example 3E.

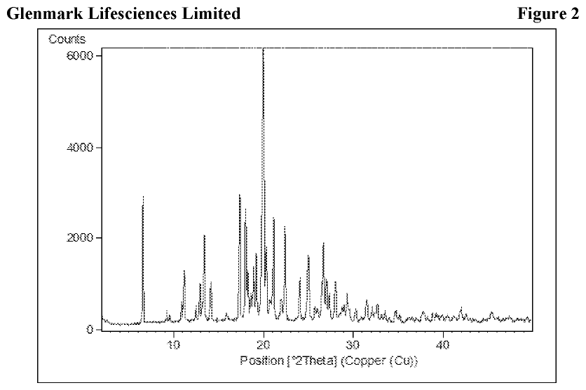

### DETAILED DESCRIPTION OF THE INVENTION

The present invention provides a process for the preparation of edoxaban tosylate monohydrate, a compound of formula I, comprising:

a) reacting a compound of formula V with a compound of formula VI in the presence of a base and a solvent to obtain a compound of formula IV, wherein the solvent is dimethyl sulfoxide;

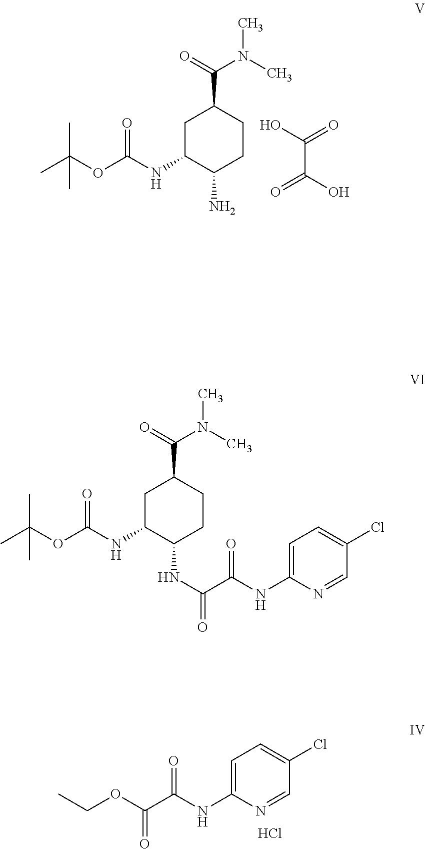

b) reacting the compound of formula IV obtained in step a) with a deprotecting reagent to obtain a compound of formula II or salt thereof,

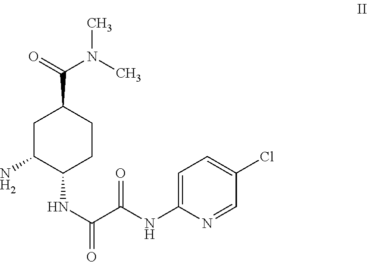 [SMILES: CN(C)C(=O)[C@H]1CC[C@H](NC(=O)C(=O)Nc2ccc(Cl)cn2)[C@H](N)C1]

c) reacting the compound of formula II or salt thereof obtained in step b) with a compound of formula III in the presence of a base to obtain a compound of formula I-A;

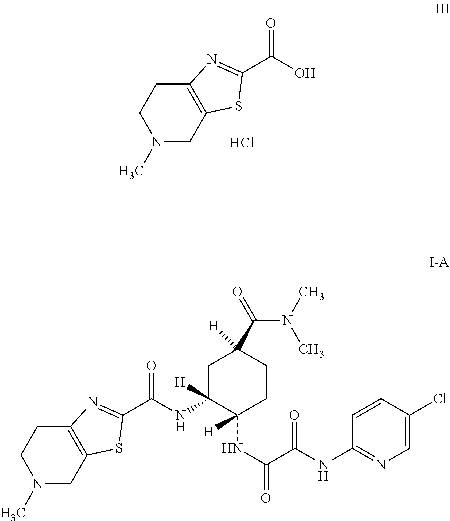

d) reacting compound of formula I-A obtained in step c) with para toluene sulfonic acid in a solvent to obtain compound of formula I; and
e) optionally, recrystallizing the compound of formula I with solvent.

In one embodiment, in step a) the base is added in one lot.

The addition of base in one lot means the entire amount of base is added all at once in a single portion and is not added in parts.

In one embodiment, in step a) the base is an amine and/or nitrogen-containing heterocyclic compounds.

In one embodiment, in step a) the base is selected from the group consisting of triethyl amine, pyridine, alkane amines, such as methylamine, diisopropylamine; imidazole, benzimidazole, histidine, guanidine phosphazene, n-methyl morpholine, morpholine, n-methylpyrolidine and the like.

In one embodiment, in step a), the base is triethyl amine.

In one embodiment, the base used in step a) is added in one lot.

In one embodiment, in step a), the base, triethyl amine is not added in parts.

In one embodiment in step a), the reaction is carried out at a temperature in the range of 40-80° C.

In one embodiment in step a), the reaction is carried out for a period of 1-10 hours.

In one embodiment in step a), the reaction is carried out at a temperature in the range of 50-60° C. for about 2-8 hours.

In one embodiment compound of formula IV is obtained in at least 90% yield with respect to compound of formula V.

In one embodiment compound of formula IV is obtained in a purity of at least 99% w/w as determined by HPLC.

In one embodiment, in step b) the deprotecting reagent is selected from ethyl acetate hydrochloride, hydrochloric acid, isopropyl alcohol HCl, methyl alcohol HCl, 1,4-dioxane HCl, ethanol HCl, isopropyl ether HCl, n-propanol HCl and the like.

In one embodiment, in step b) the deprotecting agent is hydrochloric acid.

In one embodiment, in step b) the deprotection is carried out in a solvent selected from the group consisting of ethyl acetate, acetone, isopropyl alcohol, dioxane, methanol, dimethyl sulfoxide, tetrahydrofuran, toluene, xylene, methylene dichloride, dimethyl formamide and the like.

In one embodiment, the deprotecting agent used in step b) is selected from the group consisting of ethyl acetate hydrochloride in ethyl acetate solvent, hydrochloric acid in dioxane solvent and hydrochloric acid in acetone solvent.

In embodiment, in step b) the deprotection is carried out using hydrochloric acid in acetone solvent.

In one embodiment, in step b) the compound of formula II obtained in step b) is a dihydrochloride salt, a compound of formula II-A

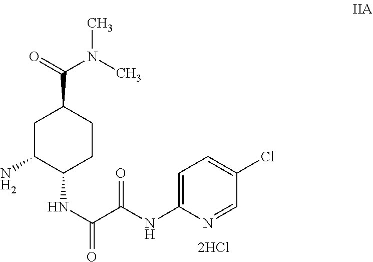 [SMILES: CN(C)C(=O)[C@H]1CC[C@H](NC(=O)C(=O)Nc2ccc(Cl)cn2)[C@H](N)C1]

In one embodiment, in step c), the reaction may be carried out in presence of a solvent selected from the group consisting of dioxane, methylene dichloride, ethyl acetate, dimethyl formamide, tetrahydrofuran, dimethyl sulfoxide.

In one embodiment, in step c) the reaction is carried out in presence of methylene dichloride solvent.

In one embodiment, in step c) the base is an amine and/or nitrogen-containing heterocyclic compounds.

In one embodiment, in step c) the base is selected from the group consisting of triethyl amine, diisopropylethyl amine, pyridine, alkane amines, such as methylamine, diisopropylamine, imidazole, benzimidazole, histidine, guanidine phosphazene bases, n-methyl morpholine, morpholine, n-methylpyrolidine and the like.

In one embodiment, the base is either triethyl amine or diisopropyl amine.

In one embodiment, in step c) an activating agent may be used.

In one embodiment, the activating agent may be selected from the group consisting of carbodiimides; 1-hydroxybenzotriazole based or 1-hydroxy-7-azabenzotriazole based phosphonium and uronium salts; halouronium and halophosphonium salts; benzotriazine based uronium salts and phosphates; N-acylimidazoles; N-acyltriazoles and the like.

In one embodiment, the carbodiimide activating agent may be selected from the group consisting of N, N′-dialkylcarbodiimides such as N, N′-dicyclohexylcarbodiimide (DCC) or diisopropylcarbodiimide (DIPC), 1-ethyl-3-(3-dimethylaminopropyl)-carbodiimide (EDC) and the like.

In one embodiment, 1-hydroxybenzotriazole and 1-hydroxy-7-azabenzotriazole based phosphonium salt may be selected from the group consisting of benzotriazol-1-yl-N-oxy-tris(dimethylamino)phosphonium hexafluorophosphate (BOP), benzotriazol-1-yl-N-oxy-tris(pyrrolidino)phosphonium hexafluorophosphate (PyBOP), 7-azabenzotriazol-1-yl-N-oxy-tris(pyrrolidino)phosphonium hexafluorophosphate (PyAOP) and the like.

In one embodiment, in step c) the coupling agent is N, N′-dicyclohexylcarbodiimide (DCC) or EDC.HCl

In one embodiment, in step c) an additive may be used.

In one embodiment, the additive may be selected from the group consisting of 1-hydroxybenzotriazole (HOBt), 1-hydroxy-7-azabenzotriazole (HOAt), 3,4-dihydro-3-hydroxy-4-oxo-1,2,3-benzotriazine (DHOBt) and the like.

In one embodiment, step c) is carried out in presence of dicyclohexylcarbodiimide (DCC) and 1-hydroxybenzotriazole (HOBt).

In one embodiment, the compound of formula I-A may be optionally purified by solvent selected from the group consisting of methanol, ethanol, isopropyl alcohol and n-butanol, water or mixtures thereof.

In one embodiment, in step d) the compound of formula I-A and para toluene sulfonic acid is suspended in a solvent selected from the group consisting of acetone, dimethyl sulfoxide, isopropyl alcohol, methanol, ethanol, water or mixture thereof.

In one embodiment, in step d) the amount of para toluene sulfonic acid added is 1.27-1.3 molar equivalent with respect to edoxaban, a compound of formula I-A.

In one embodiment, in step d) the para toluene sulfonic acid is added in one lot.

In one embodiment, in step d) the compound of formula I-A and para toluene sulfonic acid are suspended in a solvent and heated to about 40° C. to about 70° C.

In one embodiment, in step d) the compound of formula I-A and para toluene sulfonic acid is suspended in a solvent and heated to about 55° C. to about 65° C. for a period of about 1-2 hours, followed by filtration.

In one embodiment, the filtrate is cooled to obtain the compound of formula I.

In one embodiment, no water is added to the filtrate to obtain the compound of formula I.

In one embodiment, in step e) the recrystallization of compound of formula I is carried out in a solvent selected from the group consisting of acetone, dimethyl sulfoxide, isopropyl alcohol, methanol, water or mixture thereof.

In one embodiment, the present invention provides a process for the preparation of edoxaban tosylate monohydrate, a compound of formula I comprising:

a) reacting a compound of formula V with a compound of formula VI in the presence of triethyl amine and dimethyl sulfoxide to obtain a compound of formula IV;
b) reacting the compound of formula IV with ethyl acetate hydrochloride or hydrochloric acid to obtain a compound of formula II-A;
c) reacting the compound of formula II-A with a compound of formula III in the presence of a base to obtain compound of formula I-A;
d) reacting the compound of formula I-A with para toluene sulfonic acid in a solvent to obtain compound of formula I; and
e) optionally, recrystallizing the compound of formula I with a solvent.

In one embodiment, the present invention provides a process for the preparation of edoxaban tosylate monohydrate, a compound of formula I comprising:

a) reacting a compound of formula V with a compound of formula VI in the presence of triethyl amine and dimethyl sulfoxide to obtain a compound of formula IV;
b) reacting the compound of formula IV with hydrochloric acid in acetone to obtain a compound of formula II-A;
c) reacting the compound of formula II-A with a compound of formula III in the presence of a base to obtain compound of formula I-A;
d) reacting the compound of formula I-A with para toluene sulfonic acid in a solvent to obtain compound of formula I; and
e) optionally, recrystallizing the compound of formula I with a solvent.

In one embodiment, the present invention provides a process for the preparation of edoxaban tosylate monohydrate, a compound of formula I comprising:

a) reacting a compound of formula V with a compound of formula VI in the presence of base and a non-nitrile solvent to obtain compound of formula IV;
b) reacting the compound of formula IV, with a deprotecting reagent to obtain compound of formula II or salt thereof,
c) reacting compound of formula II or salt thereof with compound of formula III in the presence of a base to obtain compound of formula I-A;
d) reacting compound of formula I-A with para toluene sulfonic acid in a solvent to obtain compound of formula I; and
e) optionally, recrystallization of compound of formula I with solvent.

In one embodiment, in step a) the non-nitrile solvent is selected from the group consisting of acetone, dioxane, isopropyl alcohol, tetrahydrofuran, toluene, dimethylformamide, dimethyl acetamide, dimethyl sulfoxide.

In one embodiment, the present invention provides a compound of formula II-A

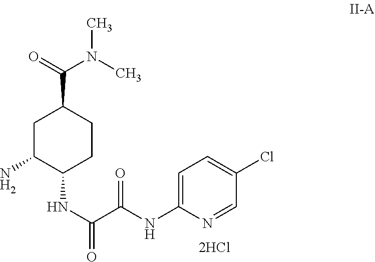 [SMILES: CN(C)C(=O)[C@H]1CC[C@H](NC(=O)C(=O)Nc2ccc(Cl)cn2)[C@H](N)C1]

characterized by 1H NMR (400 MHz, dimethyl sulfoxide, d6): 1.35-1.44, 1.65-1.78, 2.01-2.10, 2.76, 3.04, 3.32, 3.71, 3.85, 7.68, 7.98-8.05, 8.42-8.45, 8.94, 10.34.

In one embodiment, the present invention provides a process for the preparation of compound of formula II-A comprising:

a) reacting a compound of formula V with a compound of formula VI in the presence of a base and a solvent to obtain a compound of formula IV, wherein the solvent is dimethyl sulfoxide; and
b) reacting the compound of formula IV with ethyl acetate hydrochloride or hydrochloric acid to obtain a compound of formula II-A.

In one embodiment, the present invention provides a process for the preparation of compound of formula II-A comprising:

a) reacting a compound of formula V with a compound of formula VI in the presence of a base and a solvent to obtain a compound of formula IV, wherein the solvent is dimethyl sulfoxide; and
b) reacting the compound of formula IV with acetone and hydrochloric acid to obtain a compound of formula II-A.
HPLC method: High performance liquid chromatography (HPLC) was performed with the conditions described below for detecting purity:
Column: Inertsil ODS 3V 250×4.6 mm, 5μ, Column temperature: 30° C., Mobile phase: A=Buffer (100%), B=Methanol:Acetonitrile (60:40, v/v), Diluent: Water:Acetonitrile (90:10 v/v); Flow Rate: 1.0 mL/minute, Detection wavelength: UV 230 nm, Injection volume: 20 μL, Buffer: 0.1% Perchloric acid in water.

The examples that follow are provided to enable one skilled in the art to practice the invention and are merely illustrative of the invention. The examples should not be read as limiting the scope of the invention.

### EXAMPLES

### Example 1A: Preparation of Carbamic Acid, N-[(1R, 2S, 5S)-2-[[2-[(5-chloro-2-pyridinyl) amino]-2-oxoacetyl] amino]-5-[(dimethylamino) carbonyl]cyclohexyl]-1, 1-dimethylethyl Ester (Compound of Formula IV)

To a solution of t-Butyl {(1R, 2S, 5S)-2-amino-5 [(dimethylamino) carbonyl]cyclohexyl} carbamate oxalate (compound of formula V, 15 g) in dimethyl sulfoxide, triethyl amine (18.5 g) was added and the reaction mass was heated to about 50° C. to 55° C. Ethyl 2-((chloropyridin-2-yl) amino)-2-oxoacetate hydrochloride (compound of formula VI, 12.7 g) was to the reaction mass. The reaction mass was then stirred at about 55° C. to 60° C. for about 2 to about 6 hours. The reaction mass was then cooled to about 25° C. to 30° C. Water was then added to the reaction mass under stirring and the reaction mass was further cooled to about 0° C. to 5° C. and stirred for 120 minutes. The reaction mass was then filtered to obtain the product. The product obtained was washed with water and dried at about 45° C. to 50° C. for about 12 hours to obtain the compound of formula IV. Yield: 18 g.

### Example 1B: Preparation of Carbamic Acid, N-[(1R, 2S, 5S)-2-[[2-[(5-chloro-2-pyridinyl) amino]-2-oxoacetyl] amino]-5-[(dimethylamino) carbonyl]cyclohexyl]-1, 1-dimethylethyl Ester (Compound of Formula IV)

To a solution of t-Butyl {(1R, 2S, 5S)-2-amino-5 [(dimethylamino) carbonyl]cyclohexyl} carbamate oxalate (compound of formula V, 10 g) in dimethyl sulfoxide (50 mL), triethyl amine (13.46 g) was added and the reaction mass was heated to about 55° C. to 60° C. Ethyl 2-((chloropyridin-2-yl) amino)-2-oxoacetate hydrochloride (compound of formula VI, 7.77 g), was then added to the reaction mass and flushed with DMSO (10 mL). The reaction mass was then stirred at about 55° C. to 60° C. for about 5 hours, cooled to about 25° C. to 30° C. and stirred for about 12 hrs. After completion, water was added to the reaction mass under stirring and stirred for 180 minutes. Filtered the product, washed with water and dried at about 45° C. to 50° C. for about 12 hours to obtain the compound of formula IV. Yield: 11.10 g (89.5%); HPLC purity: 99.55%

### Example 2A: Preparation of Ethanediamide, N¹-[(1S, 2R, 4S)-2-amino-4-[(dimethylamino) carbonyl] cyclohexyl]-N²-(5-chloro-2-pyridinyl)-, hydrochloride (1:2) (Compound of Formula II-A)

The compound of formula IV (28.0 g) was added to ethyl acetate and ethyl acetate hydrochloride (8-10%) was then added to it. The reaction mass was heated to about 60° C. to 70° C. for about 3 hours. The reaction mass was then cooled to about 25° C. to 30° C. The reaction mass was then filtered to obtain the product. The product obtained was washed with ethyl acetate and dried at about 45° C. to 50° C. for about 12 hours to get compound of formula II-A. Yield: 26.0 g; HPLC purity: 99.0%

IR (KBr, cm-¹): 3400, 3223, 2944, 1693, 1634, 1560, 1531, 1402, 1239, 1058, 852, 774, 784.

¹H-NMR (400 MHz, dimethyl sulfoxide, d6): 1.35-1.44, 1.65-1.78, 2.01-2.10, 2.76, 3.04, 3.32, 3.71, 3.85, 7.68, 7.98-8.05, 8.42-8.45, 8.94, 10.34.

### Example 2B: Preparation of Ethanediamide, N¹-[(1S, 2R, 4S)-2-amino-4-[(dimethylamino) carbonyl] cyclohexyl]-M-(5-chloro-2-pyridinyl)-, hydrochloride (1:2) (Compound of Formula II-A)

The compound of formula IV (10.0 g) was added to isopropyl alcohol and isopropyl alcohol hydrochloride was then added to it. The reaction mass was heated to about 60° C. to 70° C. for about 5 hours. The reaction mass was then cooled to about 25° C. to 30° C. The reaction mass was then filtered to obtain the product. The product obtained was washed with isopropyl alcohol and dried at about 45° C. to 50° C. for about 12 hours to get compound of formula II-A. Yield: 70.0 g; HPLC purity: 98.0%

### Example 2C: Preparation of Ethanediamide, N¹-[(1S, 2R, 4S)-2-amino-4-[(dimethylamino) carbonyl] cyclohexyl]-M-(5-chloro-2-pyridinyl)-, hydrochloride (1:2) (Compound of Formula II-A)

The compound of formula IV (10.0 g) was added to 1, 4-dioxane and concentrated hydrochloric acid was then added to it. The reaction mass was then stirred for about 1 hour at about 25° C. to 30° C. The reaction mass was then diluted with acetone and maintained as it is for about 1 hour. The reaction mass was then filtered to obtain the product and dried at about 45° C. to 50° C. for about 12 hours to get compound of formula II-A. Yield: 9.3 g; HPLC purity: 99.67%

### Example 2D: Preparation of Ethanediamide, N¹-[(1S, 2R, 4S)-2-amino-4-[(dimethylamino) carbonyl] cyclohexyl]-M-(5-chloro-2-pyridinyl)-, hydrochloride (1:2) (Compound of Formula II)

The compound of formula IV (20.0 g) as obtained from example 1B was added to acetone (200 mL) and concentrated hydrochloric acid (40 mL) was then added to it. The reaction mass was then stirred for about 2 hours at about 25° C. to 30° C. After completion, reaction mass was filtered and dried at about 45° C. to 50° C. in vacuum tray dryer for about 6-7 hours to get compound of formula II-A. Yield: 18.40 g (97.66%); HPLC purity: 99.61%

### Example 3A: Preparation of Ethanediamide, N¹-(5-chloro-2-pyridinyl)-N²-[(1S, 2R,4S)-4-[(dimethylamino)carbonyl]-2-[[(4,5,6,7-tetrahydro-5-methylthiazolo [5,4,c]pyridin-2-yl)carbonyl]amino]cylohexyl]-,4-methylbenzenesulfonate, hydrate (1:1:1) (Compound of Formula I)

The compound of formula II-A (50.0 g) was added to 1, 4-dioxane and the obtained reaction mass was cooled to about 0° C. to 5° C. Triethyl amine (50.0 g), 5-Methyl-4, 5, 6, 7-tetrahydrothiazolo [5,4-c]pyridine-2-carboxylic acid hydrochloride (compound of formula III, 34.0 g), 1-hydroxybenzotriazole (HOBt, 20.0 g) and 1-ethyl-3-(3-dimethylaminopropyl) carbodiimide hydrochloride (EDC.HCl, 35.0 g) was added to the reaction mass. The temperature of the reaction mass was then raised to about 25° C. to 30° C. and the reaction mass was stirred for about 18 hours. Water was added to the reaction mass and the reaction mass was cooled to about 0° C. to 5° C. The reaction mass was then filtered to obtain the product (compound of formula I-A). The product obtained was washed with water. The obtained product was then added to dimethyl sulfoxide-water mixture and p-toluene sulfonic acid (17.33 g) was added to it and the reaction mass was stirred at about 25° C. to 30° C. The reaction mass was then heated to about 60° C. to 70° C. to get clear solution. The obtained clear solution was stirred for about 60 min and filtered hot to remove undissolved impurities. The filtrate was then cooled to about 25° C. to 30° C. and further to about 0° C. to 5° C. and maintained at same temperature for about 60 min. The reaction mass was then filtered to obtain the product. The product obtained dried at about 50° C. to 60° C. for about 12 hours to get compound of formula I. Yield: 100.0 g, HPLC purity: 99.25%

### Example 3B: Preparation of Ethanediamide, N¹-(5-chloro-2-pyridinyl)-N²-[(1S, 2R,4S)-4-[(dimethylamino)carbonyl]-2-[[(4,5,6,7-tetrahydro-5-methylthiazolo [5,4,c]pyridin-2-yl)carbonyl]amino]cyclohexyl]-4-methyl benzenesulfonate, hydrate (1:1:1) (Compound of Formula I)

The compound of formula II-A (10.0 g) was added to 1, 4-dioxane and the obtained reaction mass was cooled to about 0° C. to 5° C. Triethyl amine (12.42 g), 5-Methyl-4, 5,6,7-tetrahydrothiazolo[5, 4-c]pyridine-2-carboxylic acid hydrochloride (compound of formula III, 6.94 g), 1-hydroxybenzotriazole (HOBt, 4.0 g) and 1-ethyl-3-(3-dimethylaminopropyl) carbodiimide hydrochloride (EDC.HCl, 7.1 g) was added to the reaction mass. The temperature of the reaction mass was then raised to about 25° C. to 30° C. and the reaction mass was stirred for about 18 hours. Water was added to the reaction mass and the reaction mass was cooled to about 0° C. to 5° C. The reaction mass was then filtered to obtain the product (compound of formula I-A). The obtained product compound of formula I-A was then added to isopropyl alcohol-water mixture and p-toluene sulfonic acid (3.46 g) was added to it. The reaction mass was then heated to about 60° C. to 70° C. to get clear solution. The obtained clear solution was stirred for about 60 min and hot filtered to remove undissolved impurities. The filtrate was then cooled to about 25° C. to 30° C. and further to about 0° C. to 5° C. and maintained at same temperature for about 60 minutes. The reaction mass was then filtered to obtain the product. The product obtained was dried at about 50° C. to 60° C. for about 12 hours to get compound of formula I. Yield: 60%, HPLC purity: 99.25%

### Example 3C: Preparation of Ethanediamide, N¹-(5-chloro-2-pyridinyl)-N²-[(1S, 2R,4S)-4-[(dimethylamino)carbonyl]-2-[[(4,5,6,7-tetrahydro-5-methylthiazolo [5,4,c]pyridin-2-yl)carbonyl]amino]cylohexyl]-4-methylbenzenesulfonate, hydrate (1:1:1) (Compound of Formula I)

The compound of formula II-A (45.0 g) was added to 1, 4-dioxane and the obtained reaction mass was cooled to about 0° C. to 5° C. Triethyl amine (44.97 g), 5-Methyl-4, 5, 6,7-tetrahydrothiazolo[5,4-c]pyridine-2-carboxylic acid hydrochloride (compound of formula III, 31.25 g), 1-hydroxybenzotriazole (HOBt, 18.06 g) and 1-ethyl-3-(3-dimethylaminopropyl) carbodiimide hydrochloride (EDC.HCl, 32.0 g) was added to the reaction mass. The temperature of the reaction mass was then raised to about 25° C. to 30° C. and the reaction mass was stirred for about 18 hours. Water was added to the reaction mass and the reaction mass was cooled to about 0° C. to 5° C. The reaction mass was then filtered to obtain the product (compound of formula I-A). The obtained product, compound of formula I-A was then added to methanol-water mixture and p-toluene sulfonic acid (15.6 g) was added to it. The reaction mass was then heated to about 50° C. to 60° C. to get clear solution. The obtained clear solution was stirred for about 60 minutes and hot filtered to remove undissolved impurities. The filtrate was then cooled to about 25° C. to 30° C. and further to about 0° C. to 5° C. and maintained at same temperature for about 60 minutes. The reaction mass was then filtered to obtain the product. The product obtained was dried at about 50° C. to 60° C. for about 12 hours to get compound of formula I. Yield: 45.0 g, HPLC purity 99.1%.

### Example 3D: Preparation of Ethanediamide, N¹-(5-chloro-2-pyridinyl)-N²-[(1S, 2R,4S)-4-[(dimethylamino)carbonyl]-2-[[(4,5,6,7-tetrahydro-5-methylthiazolo [5,4,c]pyridin-2-yl)carbonyl]amino]cylohexyl]-4-methylbenzenesulfonate, hydrate (1:1:1) (Compound of Formula I)

The compound of formula II-A (10.0 g) was added to methylene dichloride and the obtained reaction mass was cooled to about 0° C. to 5° C. Triethyl amine (11.16 g), 5-Methyl-4,5,6,7-tetrahydrothiazolo[5,4-c]pyridine-2-carboxylic acid hydrochloride (compound of formula III, 6.96 g), 1-hydroxybenzotriazole (HOBt, 3.67 g) and 1-ethyl-3-(3-dimethylaminopropyl) carbodiimide hydrochloride (EDC.HCl, 6.52 g) was added to the reaction mass. The temperature of the reaction mass was then raised to about 25° C. to 30° C. and the reaction mass was stirred for about 18 hours. Water was added to the reaction mass and the reaction mass was stirred and the layers were separated. The organic layer was washed with brine solution. The solvent was distilled out from the organic layer to obtain the product (compound of formula I-A). The obtained product compound of formula I-A was then added to isopropyl alcohol-water mixture and p-toluene sulfonic acid (3.46 g) was added to it. The reaction mass was then heated to about 60° C. to 70° C. to get clear solution. The obtained clear solution was stirred for about 60 min and hot filtered to remove undissolved impurities. The filtrate was then cooled to about 25° C. to 30° C. and further to about 0° C. to 5° C. and maintained at same temperature for about 60 minutes. The reaction mass was then filtered to obtain the product. The product obtained was dried at about 50° C. to 60° C. for about 12 hours to get compound of formula I. Yield: 10.0 g, HPLC purity 99.4%.

### Example 3E: Preparation of Edoxaban Free Base (Compound of Formula I-A) Directly from Carbamic Acid, N-[(1R, 2S, 5S)-2-[[2-[(5-chloro-2-pyridinyl) amino]-2-oxoacetyl] amino]-5-[(dimethylamino) carbonyl] cyclohexyl]-1, 1-dimethylethyl Ester (Compound of Formula IV)

The compound of formula IV (25.0 g) obtained from example 1B was added to acetone (250 mL) and concentrated hydrochloric acid (50 mL) was then added to it. The reaction mass was then stirred for about 2 hours at about 25° C. to 30° C. After completion of the reaction mass, filtered the slurry mass, washed with acetone and suck dried. The obtained wet cake was suspended in methylene dichloride (250 mL) and added 5-Methyl-4,5,6,7-tetrahydrothiazolo[5,4-c]pyridine-2-carboxylic acid hydrochloride (compound of formula III, 15.05 g, diisopropyl ethyl amine (34.5 g), N,N-dicyclohexyl carbodiimide (DCC) (16.51 g) and 1-hydroxybenzotriazole (HOBt, 3.62 g). The reaction mass was heated to about 35° C. to 40° C. for about 5 hours. After completion, the reaction mass was cooled to about 0° C. to 5° C. for about 1 hour, filtered. The combined filtrate was washed with 5% aq. NaHCO₃ followed by brine solution. The solvent was distilled to obtain the crude product (compound of formula I-A) to which methanol (200 mL) was added and heated to 60° C. to 65° C. for about 1 hour then cooled to 25° C. to 30° C. for 1 hour, filtered, washed with methanol to obtained wet cake which was dried in tray drier at 40° C. to 45° C. to obtain compound of formula I-A (24.90 g, 85.13%), HPLC purity 99.47.

### XRPD Table for Edoxaban Free Base, Compound of Formula I-A:

| Pos. | d-spacing | Rel. Int. |
|---|---|---|
| [°2Th.] | [Å] | [%] |
| 6.62 | 13.33 | 49.05 |
| 9.26 | 9.55 | 4.61 |
| 9.58 | 9.23 | 2.78 |
| 10.89 | 8.12 | 7.02 |
| 11.16 | 7.92 | 20.03 |
| 12.48 | 7.08 | 6.13 |
| 12.91 | 6.85 | 14.75 |
| 13.18 | 6.71 | 7.26 |
| 13.41 | 6.60 | 33.38 |
| 14.14 | 6.26 | 14.84 |
| 15.87 | 5.58 | 2.76 |
| 17.35 | 5.11 | 48.75 |
| 18.02 | 4.92 | 43.36 |
| 18.25 | 4.85 | 18.84 |
| 18.58 | 4.77 | 10.11 |
| 18.91 | 4.69 | 20.74 |
| 19.22 | 4.61 | 25.78 |
| 19.85 | 4.47 | 77.01 |
| 19.99 | 4.44 | 100.00 |
| 20.30 | 4.37 | 27.85 |
| 20.66 | 4.29 | 7.37 |
| 21.11 | 4.20 | 39.50 |
| 21.98 | 4.04 | 8.17 |
| 22.40 | 3.96 | 36.18 |
| 24.10 | 3.69 | 16.51 |
| 25.05 | 3.55 | 25.20 |
| 25.77 | 3.45 | 4.81 |
| 26.03 | 3.42 | 3.98 |
| 26.73 | 3.33 | 29.62 |

### Example 3F: The Above Example May be Repeated Using EDC. HCl Instead of DCC

### Example 4A: Recrystallization of Edoxaban Tosylate Monohydrate

The compound of formula I (10.0 g) as obtained in Example 3A-3D was added to isopropyl alcohol-water mixture. The reaction mass was then heated to about 60° C. to 70° C. and was stirred for about 60 minutes and hot filtered to remove undissolved impurities. The filtrate was then cooled to about 25° C. to 30° C. for about 60 minutes. The reaction mass was then filtered to obtain the product. The product obtained was washed with isopropyl alcohol, water mixture. Then the product was dried at about 50° C. to 60° C. for about 12 hours to get compound of formula I. Yield: 9.5 g, HPLC purity: 99.67%

### Example 4B: Recrystallization of Edoxaban Tosylate Monohydrate

The compound of formula I (10.0 g) as obtained in Example 3A-3D was added to dimethyl sulfoxide-water mixture. The reaction mass was then heated to about 60° C. to 70° C. and was stirred for about 60 minutes and hot filtered and washed with dimethyl sulfoxide. The filtrate was again heated to 70° C. and stirred for about 30 minutes and then cooled to 50° C. and stirred for 120 min. It was further cooled to about 0° C.-5° C. and stirred for 60 min. The product was filtered, wet cake was washed with water and dried at about 50° C. to 60° C. for 12.0 hours to get edoxaban tosylate monohydrate; yield 9.5 g, HPLC purity 99.14%.

### Example 4C: Recrystallization of Edoxaban Tosylate Monohydrate

The compound of formula I (10.0 g) as obtained in Example 3A-3D was added to methanol-water mixture. The reaction mass was then heated to about 60° C. to 65° C. and was stirred for about 60 minutes and hot filtered and washed with dimethyl sulfoxide. The filtrate was cooled to about 25° C.-30° C. and further to about 0° C.-5° C. and stirred for 60 min. The product was filtered, wet cake was washed with water and dried at about 50° C. to 60° C. for 12.0 hours to get edoxaban tosylate monohydrate; yield 9.0 g, HPLC purity 99.18%.

### Example 4D: Recrystallization of Edoxaban Tosylate Monohydrate

The compound of formula I (13.0 g) as obtained in Example 3A-3D was added to acetone (97.5 mL), water (97.5 mL) mixture. The reaction mass was then heated to about 50° C. to 55° C. and was stirred for about 30 minutes and filtered hot and washed with acetone and water mixture. The filtrate was cooled to about 25° C.-30° C. and further to about 5° C. to 10° C. and stirred for 120 min. The product was filtered, wet cake was washed with water and dried at about 45° C. to 50° C. for 12.0 hours to get edoxaban tosylate monohydrate; yield 11.20 g (86.0%), HPLC purity: 99.91%

### Example 4E: Preparation of Ethanediamide, N¹-(5-chloro-2-pyridinyl)-N²-[(1S, 2R,4S)-4-[(dimethylamino)carbonyl]-2-[[(4,5,6,7-tetrahydro-5-methylthiazolo [5,4,c]-pyridin-2-yl)carbonyl]amino]cylohexyl]-,4-methyl benzene sulfonate, hydrate (1:1:1) (Compound of Formula I)

The compound of formula I-A (20.0 g) as obtained in Example 3E-3F was added to acetone (125 mL), water (125 mL) mixture and p-toluene sulfonic acid 8.30 g) was added to it and flushed with 50% aq. (50 mL). The reaction mass was then heated to about 50° C. to 60° C. to get clear solution. The obtained clear solution was stirred for about 60 min and hot filtered and washed with acetone and water mixture. The filtrate was then cooled to about 5° C. to 10° C. and maintained at same temperature for about 240 minutes. The reaction mass was then filtered and dried at about 45° C. to 50° C. for about 12 hours to get compound of formula I. Yield: 22.0 g (82.0%), HPLC purity 99.94%

### XRPD Table for Edoxaban Tosylate Monohydrate, Compound of Formula I:

| Pos. | d-spacing | Rel. Int. |
|---|---|---|
| [°2Th.] | [Å] | [%] |
| 2.73 | 32.30 | 9.99 |
| 5.44 | 16.23 | 38.20 |
| 8.15 | 10.83 | 28.00 |
| 10.86 | 8.14 | 90.35 |
| 11.78 | 7.50 | 4.12 |
| 12.76 | 6.93 | 5.06 |
| 13.57 | 6.52 | 31.21 |
| 15.10 | 5.86 | 25.07 |
| 16.55 | 5.35 | 5.25 |
| 16.95 | 5.23 | 37.33 |
| 17.18 | 5.16 | 10.11 |
| 17.62 | 5.03 | 17.99 |
| 18.43 | 4.81 | 1.51 |
| 19.04 | 4.66 | 17.12 |
| 19.62 | 4.52 | 4.01 |
| 20.59 | 4.31 | 27.63 |
| 21.09 | 4.21 | 61.47 |
| 21.25 | 4.17 | 51.68 |
| 22.81 | 3.89 | 100.00 |
| 23.58 | 3.77 | 40.58 |
| 24.70 | 3.60 | 3.01 |
| 25.16 | 3.53 | 12.46 |
| 26.02 | 3.42 | 42.79 |
| 26.77 | 3.32 | 13.63 |
| 27.33 | 3.26 | 59.60 |
| 27.62 | 3.22 | 33.62 |
| 28.56 | 3.12 | 5.15 |
| 28.96 | 3.08 | 19.92 |
| 30.12 | 2.96 | 33.19 |

### Example 5: Preparation of Carbamic Acid, N-[(1R, 2S, 5S)-2-[[2-[(5-chloro-2-pyridinyl)amino]-2-oxoacetyl] amino]-5-[(dimethylamino) carbonyl] cyclohexyl]-1,1-dimethylethyl Ester (Compound of Formula IV)

To a solution of t-Butyl {(1R, 2S, 5S)-2-amino-5 [(dimethylamino) carbonyl]cyclohexyl} carbamate oxalate (compound of formula V, 10.0 g) in 1,4-dioxane, triethyl amine (12.35 g) was added and the reaction mass was heated to about 50° C. to 55° C. At this stage the reaction mass thicken due to gel deposition and stirring becomes difficult. Ethyl 2-((chloropyridin-2-yl) amino)-2-oxoacetate hydrochloride (compound of formula VI, 8.47 g) was then added to the reaction mass. The reaction mass was then stirred at about 55° C. to 60° C. for about 18 hours. The reaction mass was then cooled to about 25° C. to 30° C. Water was then added to the reaction mass under stirring and the reaction mass was stirred for 60 minutes. The reaction mass was then filtered to obtain the product. The product obtained was dried at about 45° C. to 50° C. to obtain the compound of formula IV. Yield: 5 g.

### Example 6: Preparation of Carbamic Acid, N-[(1R, 2S, 5S)-2-[[2-[(5-chloro-2-pyridinyl)amino]-2-oxoacetyl] amino]-5-[(dimethylamino) carbonyl] cyclohexyl]-1,1-dimethylethyl Ester (Compound of Formula IV)

To a solution of t-Butyl {(1R, 2S, 5S)-2-amino-5 [(dimethylamino)carbonyl]cyclohexyl}carbamate oxalate (compound of formula V, 10.0 g) in isopropyl alcohol, triethyl amine (12.34 g) was added and the reaction mass was heated to about 50° C. to 55° C. At this stage the reaction mass thickens due to gel deposition and stirring becomes difficult. Ethyl 2-((chloropyridin-2-yl) amino)-2-oxoacetate hydrochloride (compound of formula VI, 8.47 g) was then added to the reaction mass. The reaction mass was then stirred at about 55° C. to 60° C. for about 10 hours. The reaction mass was then cooled to about 25° C. to 30° C. Water was then added to the reaction mass under stirring and the reaction mass was stirred for 60 minutes. The reaction mass was then filtered to obtain the product. The product obtained was dried at about 45° C. to 50° C. for about 12 hours to obtain the compound of formula IV. Yield: 7 g.

### Comparative Example 1: Preparation of Carbamic Acid, N-[(1R, 2S, 5S)-2-[[2-[(5-chloro-2-pyridinyl) amino]-2-oxoacetyl] amino]-5-[(dimethylamino) carbonyl] cyclohexyl]-1, 1-dimethylethyl Ester (Compound of Formula IV)

To a solution of t-Butyl {(1R, 2S, 5S)-2-amino-5 [(dimethylamino) carbonyl]cyclohexyl} carbamate oxalate (compound of formula V, 25 g) in acetonitrile, triethyl amine (30.6 g) was added and the reaction mass was heated to about 50° C. to 55° C. Ethyl 2-((chloropyridin-2-yl) amino)-2-oxoacetate hydrochloride (compound of formula VI, 21.1 g) was then added to the reaction mass. At this stage the reaction mass thicken due to gel deposition and stirring becomes difficult. The reaction mass was then stirred at about 55° C. to 60° C. for about 6 hours. The reaction mass was then cooled to about 25° C. to 30° C. for about 12 hours. Water was then added to the reaction mass under stirring and the reaction mass was further cooled to about 0° C. to 5° C. and stirred for 120 minutes. The reaction mass was then filtered to obtain the product. The product obtained was washed with water and dried at about 45° C. to 50° C. for about 12 hours to obtain the compound of formula IV. Yield: 24.0 g.

1. A process for the preparation of edoxaban tosylate monohydrate, a compound of formula I comprising: 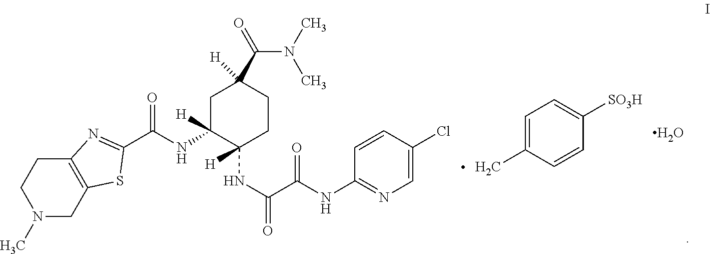
the process comprising:
a) reacting a compound of formula V with a compound of formula VI in the presence of a first base and a first solvent to obtain a compound of formula IV, wherein the solvent is dimethyl sulfoxide; 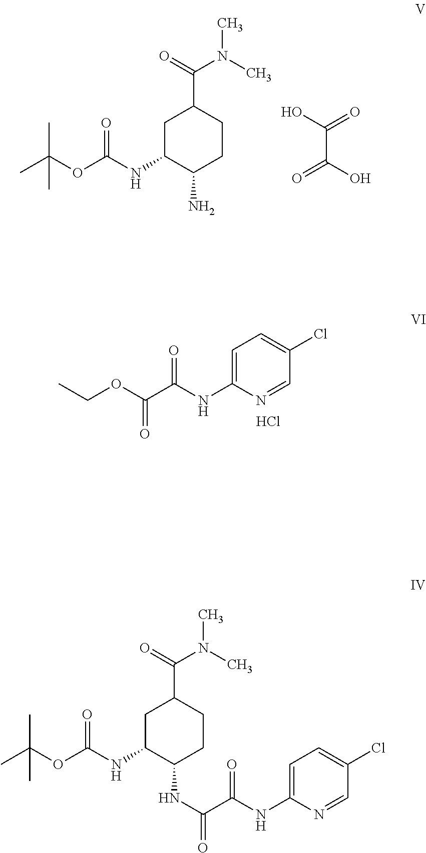
b) reacting the compound of formula IV with a deprotecting agent to obtain a compound of formula II or a salt thereof; 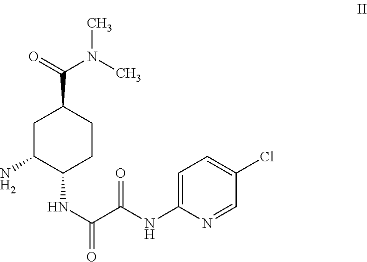 [SMILES: CN(C)C(=O)[C@H]1CC[C@H](NC(=O)C(=O)Nc2ccc(Cl)cn2)[C@H](N)C1]
c) reacting the compound of formula II or salt thereof with a compound of formula III in the presence of a second base to obtain a compound of formula I-A; 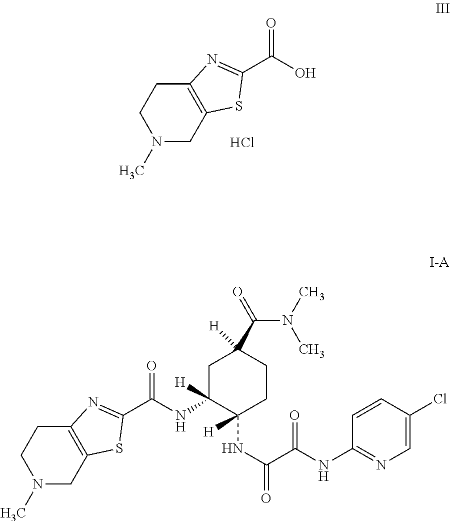
d) reacting the compound of formula I-A with para toluene sulfonic acid in a second solvent to obtain a compound of formula I; and
e) optionally, recrystallizing the compound of formula I with a third solvent.

2. The process according to claim 1, wherein the first base used in step a) is triethylamine.

3. The process according to claim 1, wherein the first base used in step a) is added in one lot.

4. The process according to claim 1, wherein the deprotecting agent used in step b) is selected from the group consisting of ethyl acetate hydrochloride in an ethyl acetate solvent, hydrochloric acid in a dioxane solvent and hydrochloric acid in an acetone solvent.

5. The process according to claim 1, wherein the compound of formula II obtained in step b) is a dihydrochloride salt, a compound of formula II-A.

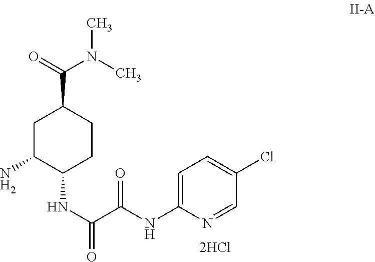 [SMILES: CN(C)C(=O)[C@H]1CC[C@H](NC(=O)C(=O)Nc2ccc(Cl)cn2)[C@H](N)C1]

6. The process according to claim 1, wherein the third base used in step c) is selected from the group consisting of triethyl amine, and diisopropyl ethyl amine.

7. The process according to claim 1, wherein the second solvent used in step d) is selected from the group consisting of acetone, dimethyl sulfoxide, isopropyl alcohol, methanol, water and mixtures thereof.

8. The process according to claim 1, wherein the third solvent used in step e) is selected from the group consisting of acetone, dimethyl sulfoxide, methanol, isopropyl alcohol, methanol, water and mixtures thereof.

9. The process according to claim 8, wherein edoxaban tosylate monohydrate is crystallized from a solvent mixture of acetone and water.
